(19) United States
(12) Patent Application Publication
(10) Pub. No.: US 2025/0378560 A1
(43) Pub. Date: Dec. 11, 2025

(54) METHOD AND DEVICE FOR PROVIDING INFORMATION ON STRAIN QUANTIFICATION

(71) Applicant: ONTACT HEALTH CO., LTD., Seoul (KR)

(72) Inventors: Seung Ah Lee, Seoul (KR); Young Taek Hong, Seoul (KR); Yeong Gul Jang, Seoul (KR)

(73) Assignee: ONTACT HEALTH CO., LTD., Seoul (KR)

(21) Appl. No.: 19/229,913

(22) Filed: Jun. 5, 2025

(30) Foreign Application Priority Data
Jun. 7, 2024 (KR) 1020240074175
Aug. 27, 2024 (KR) 1020240115280

(51) Int. Cl. G06T 7/00; A61B 8/00; A61B 8/08; G06T 7/11; G06T 7/246; G06V 10/25; G06V 10/26; G06V 10/62; G06V 10/764; G16H 30/40; G16H 50/50

(52) U.S. Cl. CPC G06T 7/0016; A61B 8/0883; A61B 8/485; A61B 8/5223; G06T 7/11; G06T 7/248; G06V 10/25; G06V 10/26; G06V 10/62; G06V 10/764; G16H 30/40; G16H 50/50; G06T 2207/10016; G06T 2207/10132; G06T 2207/20081; G06T 2207/20084; G06T 2207/30048; G06V 2201/031

(57) ABSTRACT

The present disclosure provides a method for providing information on strain quantification implemented by a processor, the method includes receiving a cardiac ultrasound image including a target heart area of an subject, determining a motion vector field for the target heart area in the received cardiac ultrasound image using a prediction model trained to segment the target heart area using the cardiac ultrasound image as an input and determine a motion vector field based on the segmented target heart area, and determining a strain quantification parameter based on the motion vector field, and the present disclosure provides a device and system using the method for providing information on strain quantification.

### CROSS-REFERENCE TO RELATED APPLICATIONS

This application claims priority to Korean Patent Application No. 10-2024-0074175, filed on Jun. 7, 2024, and to Korean Patent Application No. 10-2024-0115280, filed on Aug. 27, 2024, both of which are incorporated by reference in entirety for all purposes. The USPTO is invited to retrieve the priority documents using the provided DAS codes.

### BACKGROUND

### Field

The present disclosure relates to a method for providing information on strain quantification and a device for providing information on strain quantification using the same.

### Description of the Related Art

Strain is a technique for determining whether an organ is abnormal by measuring a strain rate of micro-muscles.

In particular, in relation to cardiovascular diagnosis, strain is used to evaluate cardiac function by measuring the movement of cardiac muscles. In this case, the strain is measured through cardiac ultrasound, and may be expressed as a percentage of how much the length changes when the muscle contracts or relaxes. Through this, the contraction and relaxation state of a cardiac target area (for example, the left ventricle) may be quantitatively analyzed.

Meanwhile, this strain quantitative analysis is essential for the diagnosis and treatment of heart disease, but when measured manually, it is time-consuming and the results may vary depending on the subjectivity of the expert. Furthermore, manual measurement of strain requires multiple steps, and errors may occur at each step.

That is, in the case of manual measurement of the strain, it may depend on the experience of experts, and there is a problem that the reliability of the analysis results is insufficient depending on the skill level of the medical staff.

Therefore, the development of an information provision system for strain quantification that can provide accurate and highly reproducible results is continuously required.

The technology that forms the background of the disclosure has been written to facilitate understanding of the present disclosure. It should not be understood that the matters described in the technology that forms the background of the disclosure exist as prior art.

### SUMMARY

Recently, various techniques for strain analysis have been proposed along with the development of artificial intelligence technology.

Meanwhile, the proposed artificial intelligence-based strain analysis techniques are mainly limited to measuring the size and volume of a left ventricle (LV) and calculating parameters such as LV ejection fraction (LVEF) to evaluate the LV contractile function, and thus may have a limited scope of application.

The inventors of the present disclosure have sought to build an artificial neural network-based prediction model capable of analyzing various parameters including not only left ventricular contractile function but also left atrium (LA) volume measurement and further left ventricular diastolic function (LVDF) evaluation.

In particular, the inventors of the present disclosure have sought to provide an information provision system capable of motion estimation and strain quantitative analysis for target heart areas such as LV or LA by constructing a single model to segment the anatomical structure of the heart and simultaneously determine the motion vector field.

Accordingly, the inventors of the present disclosure have been able to recognize that the problem of errors occurring due to multiple steps in manual strain measurement can be solved.

In relation to this, the inventors of the present disclosure have been able to recognize that motion estimation robust to image noise is possible by considering global structural features and regional feature similarities when predicting a motion vector field between adjacent frames.

As a result, the inventors of the present disclosure have been able to apply the prediction model trained to predict the motion field vector between adjacent frames for the segmented target heart area to a new information provision system.

Furthermore, the inventors of the present disclosure have been able to perform geometric modeling that enables motion estimation of the target area by defining a spline curve using a spline mathematical technique.

In this case, the inventors of the present disclosure are able to recognize that motion estimation is possible without folding by setting a region of interest (ROI) based on the intermediate layer of the inner wall of the heart structure which is the target area, and generating a spline curve.

As a result, the inventors of the present disclosure are able to accurately evaluate the movement of the cardiac muscle by providing a new information provision system based on spatiotemporal motion modeling, and thus provide not only visual information of the dysfunctional area, but also highly reliable strain quantitative information.

Accordingly, an object of the present disclosure is to provide a method, device, and system for providing information on strain quantification, configured to determine a motion vector field for a target heart area using a prediction model for a cardiac ultrasound image acquired from an object and to provide information related to strain quantification based on the motion vector field.

Objects of the present disclosure are not limited to the object mentioned above, and other objects that are not mentioned can be clearly understood by those skilled in the art from the description below.

In order to achieve the above-described objects, there is provided a method for providing information on strain quantification according to one embodiment of the present disclosure. The method is a method for providing information on strain quantification implemented by a processor, and includes: receiving a cardiac ultrasound image including a target heart area of a subject; determining a motion vector field for the target heart area in the received cardiac ultrasound image using a prediction model trained to segment the target heart area using the cardiac ultrasound image as an input and determine a motion vector field based on the segmented target heart area; and determining a strain quantification parameter based on the motion vector field.

In this case, the target heart area is at least one of a left ventricle (LV), a right ventricle (RV), a left atrium (LA), and a right atrium (RA).

According to an aspect of the present disclosure, the cardiac ultrasound image may be a video including a plurality of frames, and the prediction model may be configured to determine the motion vector field for at least one frame selected from the plurality of frames based on a frame adjacent to the at least one frame.

According to another aspect of the present disclosure, the plurality of frames may include a plurality of frames having a first resolution and a plurality of frames having a second resolution for the target heart area, and the determining of the motion vector field may include, by using the prediction model, determining a correlation for the plurality of frames having the first resolution, determining a correlation for the plurality of frames having the second resolution, integrating a motion feature based on the correlation for each of the first resolution and the second resolution, and determining the motion vector field based on the integrated motion feature.

According to still another aspect of the present disclosure, the first resolution or the second resolution may have a resolution greater than that of the remaining one, and the determining of the motion vector field based on the integrated motion feature may further include determining a feature map for a plurality of frames corresponding to the large resolution, and determining the motion vector field based on the feature map and the integrated motion feature.

According to still another aspect of the present disclosure, the plurality of frames may include a first frame for the target heart area and a second frame that is a frame before or after the first frame, and the determining of the motion vector field may include, by using the prediction model, determining a first motion vector field for the first frame, and estimating a second motion vector field for the second frame based on the first motion vector field.

According to still another aspect of the present disclosure, the determining of the motion vector field may include, by using the prediction model, determining a spline curve using a spline mathematical technique to estimate motion for the target heart area.

According to still another aspect of the present disclosure, the determining of the spline curve may further include determining a heart wall within the target heart area, determining an intermediate layer for the heart wall, expanding the intermediate layer to determine a region of interest (ROI), and obtaining a spline curve for the ROI.

According to still another aspect of the present disclosure, the determining of the spline curve may include determining a plurality of spline curve layers to obtain a spline surface including the plurality of spline curve layers.

According to still another aspect of the present disclosure, the method may further include correcting the determined spline curve.

According to still another aspect of the present disclosure, the correcting of the spline curve may further include determining a curvature for the spline curve, and correcting the spline curve by cutting the spline curve by excluding a data point, the curvature of which is equal to or greater than a predetermined level, among data points forming the spline curve.

According to still another aspect of the present disclosure, the correcting of the spline curve may further include a smoothing by assigning weight to a data point corresponding to a specific area of the target heart area in a process of generating the spline curve.

According to still another aspect of the present disclosure, the prediction model may be a model further trained to classify a cross-sectional view of the ultrasound image by using the cardiac ultrasound image as the input, and the determining of the motion vector field may further include, by using the prediction model, classifying a cross-sectional view of the received ultrasound image, segmenting the target heart area for the ultrasound image corresponding to the classified cross-sectional view, and determining a motion vector field for the target heart area, and the determining of the strain quantification parameter may further include determining a strain quantification parameter corresponding to the classified view.

According to still another aspect of the present disclosure, the method may further include outputting and providing a mask for the target heart area segmented by the prediction model.

According to still another aspect of the present disclosure, the target heart area may be LA, and the determining of the strain quantification parameter may include determining a strain curve for the LA based on the motion vector field, and determining a quantification parameter for the LA based on the strain curve.

In order to achieve the above-described objects, there is provided a device for providing information on strain quantification according to one embodiment of the present disclosure.

The device includes: a communication unit configured to receive a cardiac ultrasound image including a target heart area of a subject; and a processor functionally connected to the communication unit. In this case, the processor is configured to determine a motion vector field for the target heart area in the received cardiac ultrasound image using a prediction model trained to segment the target heart area using the cardiac ultrasound image as an input and determine a motion vector field based on the segmented target heart area, and determine a strain quantification parameter based on the motion vector field, and the target heart area is at least one of a left ventricle (LV), a right ventricle (RV), a left atrium (LA), and a right atrium (RA).

According to an aspect of the present disclosure, the cardiac ultrasound image may be a video including a plurality of frames, and the prediction model may be configured to determine the motion vector field for at least one frame selected from the plurality of frames based on a frame adjacent to the at least one frame.

According to another aspect of the present disclosure, the plurality of frames may include a plurality of frames having a first resolution and a plurality of frames having a second resolution for the target heart area, and the processor may be further configured to, by using the prediction model, determine a correlation for the plurality of frames having the first resolution, determine a correlation for the plurality of frames having the second resolution, integrate a motion feature based on the correlation for each of the first resolution and the second resolution, and determine the motion vector field based on the integrated motion feature.

According to still another aspect of the present disclosure, the first resolution or the second resolution may have a resolution greater than that of the remaining one, and the processor may be further configured to determine a feature map for a plurality of frames corresponding to the large resolution, and determine the motion vector field based on the feature map and the integrated motion feature.

According to still another aspect of the present disclosure, the plurality of frames may include a first frame for the target heart area and a second frame that is a frame before or after the first frame, and the processor may be further configured to, by using the prediction model, determine a first motion vector field for the first frame, and estimate a second motion vector field for the second frame based on the first motion vector field.

According to still another aspect of the present disclosure, the processor may be further configured to, by using the prediction model, determine a spline curve using a spline mathematical technique to estimate motion for the target heart area.

According to still another aspect of the present disclosure, the processor may be further configured to determine a heart wall within the target heart area, determine an intermediate layer for the heart wall, expand the intermediate layer to determine a region of interest (ROI), and obtain a spline curve for the ROI.

According to still another aspect of the present disclosure, the processor may be further configured to determine a plurality of spline curve layers to obtain a spline surface including the plurality of spline curve layers.

According to still another aspect of the present disclosure, the prediction model may be a model further trained to classify a cross-sectional view of the ultrasound image by using the cardiac ultrasound image as the input, and the processor may be further configured to, by using the prediction model, classify a cross-sectional view of the received ultrasound image, segment the target heart area for the ultrasound image corresponding to the classified cross-sectional view, determine a motion vector field for the target heart area, and determine a strain quantification parameter corresponding to the classified view.

According to still another aspect of the present disclosure, the target heart area may be LA, and the processor may be further configured to determine a strain curve for the LA based on the motion vector field, and determine a quantification parameter for the LA based on the strain curve.

To achieve the above-described objects, there is provided a system for providing information on strain quantification according to one embodiment of the present disclosure.

The system includes: an internal memory configured to store a cardiac ultrasound image including a target heart area of a subject, and a prediction model trained to segment the target heart area by using the cardiac ultrasound image as an input and to determine a motion vector field based on the segmented target heart area; and a processing unit configured to access the internal memory, determine the motion vector field for the target heart area in the received cardiac ultrasound image using the prediction model, and determine a strain quantification parameter based on the motion vector field, in which the target heart area is at least one of a left ventricle (LV), a right ventricle (RV), a left atrium (LA), and a right atrium (RA).

Specific details of other embodiments are included in the detailed description and drawings.

The present disclosure can provide an information provision system using an artificial neural network-based prediction model capable of analyzing various strain quantification parameters including not only a contractile function of a left ventricle but also volume measurement of a left atrium and a diastolic function evaluation of a left ventricle.

Therefore, according to the present disclosure, it is possible to overcome the limitations of conventional strain quantitative analysis methods that are limited to measuring the size and volume of the left ventricle and calculating parameters such as LV ejection fraction for evaluating the contractile function of the left ventricle.

In particular, the present disclosure can provide highly reliable information for strain quantification by providing the information provision system capable of predicting the motion vector field between adjacent frames for the cardiac ultrasound image.

Therefore, the present disclosure can overcome the limitations of conventional strain (or strain rate) analysis-based information provision systems that only consider regional features.

The present disclosure can overcome the limitations of the conventional information provision system that relies on the image quality and the experience of experts in strain analysis of the cardiac ultrasound image and requires the application of a motion noise postprocessing algorithm because it only considers regional features.

In addition, the present disclosure can set the ROI based on the intermediate layer of the inner wall of the cardiac structure which is the target area, and generate a spline curve to enable motion estimation of the cardiac structure without folding.

Therefore, the present disclosure can provide a new information provision system based on spatiotemporal motion modeling, which can accurately evaluate the movement of the cardiac muscle and provide not only visual information of the dysfunctional area but also highly accurate strain quantitative information.

Furthermore, the present disclosure can solve the problem of errors occurring in manual strain measurement by automating the procedure performed in multiple steps using a single model and enabling the determination of strain quantification parameters.

In other words, the present disclosure can contribute to the early diagnosis and good treatment prognosis of cardiopulmonary diseases by providing information on the strain quantification.

The effects according to the present disclosure are not limited to the contents exemplified above, and more diverse effects are included in the present disclosure.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a schematic diagram for explaining an information provision system using a cardiac ultrasound image according to one embodiment of the present disclosure;

FIG. 2A is a schematic diagram for explaining a device for providing information on strain quantification according to one embodiment of the present disclosure;

FIG. 2B is a schematic diagram for explaining a user device that receives information from the device for providing information on strain quantification according to one embodiment of the present disclosure;

FIGS. 3A to 3K are schematic flowcharts for explaining a method for providing information on strain quantification based on a motion vector field in the device for providing information on strain quantification according to one embodiment of the present disclosure, and exemplarily illustrate a procedure for determining a motion vector field;

FIG. 4 exemplarily illustrates a structure of a prediction model applied to various embodiments of the present disclosure;

FIG. 5 exemplarily illustrates a modeling procedure of a prediction model applied to various embodiments of the present disclosure;

FIGS. 6A to 6D illustrate the evaluation results of the prediction model used for the device for providing information on strain quantification according to various embodiments of the present disclosure in a left ventricle (LV);

FIG. 7 illustrates the evaluation results of the prediction model used for the device for providing information on strain quantification according to various embodiments of the present disclosure in a left atrium (LA); and

FIG. 8 illustrates the evaluation results of the prediction model used for the device for providing information on strain quantification according to various embodiments of the present disclosure in a right ventricle (RV).

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

The advantages and features of the present disclosure, and the methods for achieving them, will become clear with reference to the embodiments described in detail below together with the attached drawings. However, the present disclosure is not limited to the embodiments disclosed below, but may be implemented in various different forms, and the present embodiments are provided only to make the disclosure of the present disclosure complete and to fully inform a person having ordinary skill in the art to which the present disclosure belongs of the scope of the disclosure. In connection with the description of the drawings, similar reference numerals may be used for similar components.

In the present disclosure, the expressions “have”, “may have”, “include”, or “may include” indicate the presence of a given feature (for example, a component such as a number, function, operation, or part), and do not exclude the presence of additional features.

In the present disclosure, the expressions “A or B”, “at least one of A or/and B”, or “one or more of A or/and B” may include all possible combinations of the items listed together. For example, “A or B”, “at least one of A and B”, or “at least one of A or B” may all refer to (1) including at least one A, (2) including at least one B, or (3) including both at least one A and at least one B.

The expressions “first”, or “second”, used in the present disclosure may describe various components, regardless of order and/or importance, and are used only to distinguish one component from another, but do not limit the components. For example, a first user device and a second user device may represent different user devices, regardless of order or importance. For example, without departing from the scope of the rights set forth in the present disclosure, a first component may be referred to as a second component, and similarly, a second component may be referred to as a first component.

When it is stated that a component (for example, a first component) is “(operatively or communicatively) coupled with/to” or “connected to” another component (for example, a second component), it should be understood that the component may be directly coupled to the other component, or may be connected via another component (for example, a third component). Conversely, when it is stated that a component (for example, a first component) is “directly coupled to” or “directly connected to” another component (for example, a second component), it should be understood that no other component (for example, a third component) exists between the component and the other component.

The expression “configured (or set) to” as used herein may be used interchangeably with, for example, “suitable for”, “having the capacity to”, “designed to”, “adapted to”, “made to”, or “capable of”, depending on the context. The term “configured (or set) to” does not necessarily mean something that is “specifically designed to” in terms of hardware. Instead, in some contexts, the expression “a device configured to” may mean that the device is “capable of” doing something in conjunction with other devices or components. For example, the phrase “a processor configured (or set) to perform A, B, and C” can mean a dedicated processor (for example, an embedded processor) to perform those operations, or a generic-purpose processor (for example, a CPU or an application processor) that can perform those operations by executing one or more software programs stored in a memory device.

The terms used in the present disclosure are only used to describe specific embodiments and may not be intended to limit the scope of other embodiments. The singular expression may include the plural expression unless the context clearly indicates otherwise. The terms used herein, including technical or scientific terms, may have the same meaning as commonly understood by a person of ordinary skill in the art described in the present disclosure. Among the terms used in the present disclosure, terms defined in general dictionaries may be interpreted as having the same or similar meaning in the context of the related technology, and shall not be interpreted in an ideal or excessively formal meaning unless explicitly defined in the present disclosure. In some cases, even when a term is defined in the present disclosure, it cannot be interpreted to exclude the embodiments of the present disclosure.

The individual features of the various embodiments of the present disclosure may be partially or wholly coupled or combined with each other, and various technical connections and operations are possible, as can be fully understood by those skilled in the art, and each embodiment may be implemented independently of each other or may be implemented together in a related relationship.

For clarity in the interpretation of the present specification, the terms used in the present specification are defined below.

The term “cardiac ultrasound image” used in the present specification may mean an image that noninvasively visualizes the structure and function of the heart using ultrasound technology.

In this case, the cardiac ultrasound image may be a two-dimensional cardiac ultrasound image, but is not limited thereto, and may also be a three-dimensional cardiac ultrasound image.

Furthermore, the cardiac ultrasound image may be a periodic cardiac ultrasound image (or video) including a plurality of frames.

In various embodiments, the cardiac ultrasound image may be a B-mode ultrasound image, but is not limited thereto.

Meanwhile, the cardiac ultrasound image may determine the motion vector field between adjacent frames for each frame of the image according to the method for providing information on strain quantification according to one embodiment of the present disclosure.

According to another aspect of the present disclosure, the cardiac ultrasound image may be a time-series echocardiographic image having a sequence of two beats.

For example, a 2-bit unit cardiac ultrasound image may include a frame of end systole (ES) and a frame of end diastole (ED), and further may be a time-series echocardiographic image including a mid (ES-ED) frame between the end systole and end diastole, and a mid (ED-ES) frame between end diastole and end systole.

The term “target heart area” as used in the present specification may refer to an area of anatomical structures of the heart in the cardiac ultrasound image.

In one embodiment of the present disclosure, the target heart area may be an area selected from a right ventricle anterior wall, a right ventricle (RV), an anterior wall of the aorta, an aorta, a posterior wall of an aorta, a left atrium (LA), and a posterior wall of a left atrium (LA).

The term “motion vector field” used in the present specification means a vector field, which is a space expressed as a vector function, and may be a plurality of vectors that presents the direction and size according to the movement of micro-muscles of an organ within an image (or frame).

More specifically, the motion vector field in the present specification may mean vectors for each of a plurality of pixels forming the target heart area in the frame of the cardiac ultrasound image. Accordingly, strain analysis according to the global structural features of an organ may be possible by determining the motion vector field between adjacent frames.

The term “prediction model” used in the present specification may be a model trained to input the cardiac ultrasound image, segment the target heart area, and determine the motion vector field based on the segmented target heart area.

According to an aspect of the present disclosure, the prediction model may be a model trained to input a cardiac ultrasound image, classify a cross-sectional view of the ultrasound image, segment the classified target heart area, and determine a motion vector field based on the segmented target heart area.

Here, the prediction model may classify at least one cross-sectional view among a B-mode parasternal long-axis (PLAX) cross-sectional view, a B-mode parasternal short-axis (PSAX) aortic valve (AV) level cross-sectional view, a B-mode PSAX mitral valve (MV) level cross-sectional view, a B-mode apical 4-chamber (A4C) cross-sectional view, a B-mode apical 2-chamber (A2C) cross-sectional view, a B-mode apical 3-chamber (A3C) cross-sectional view, a B-mode subcostal 4-chamber cross-sectional view, a B-mode suprasternal cross-sectional view, a B-mode left ventricular outflow tract (LVOT) cross-sectional view, and a B-mode right ventricular (RV) cross-sectional view for the cardiac ultrasound image.

However, the present disclosure is not limited thereto, and the prediction model may be a model trained to classify cross-sectional views corresponding to an M-mode and a Doppler mode.

Furthermore, in various embodiments of the present disclosure, the prediction model may be trained to distinguish the direction of the cardiac ultrasound image as right or left.

That is, the prediction model may be a hybrid model that combines various modules that support the automation of the strain quantification and image processing process.

In various embodiments of the present disclosure, the prediction model may be composed of a cross-sectional view classification unit that classifies a cross-sectional view of the input cardiac ultrasound image, a target heart area segmentation unit that segments the target heart area, and a motion estimation unit that estimates the motion vector field.

However, the present disclosure is not limited thereto, and the prediction model may be composed of a more diverse combination of modules that provide information on strain quantification.

According to another embodiment of the present disclosure, the prediction model may be a model trained to estimate a motion vector field based on adjacent frames.

That is, the prediction model may learn spatiotemporal characteristics of the cardiac ultrasound image and estimate the motion vector field for each of the plurality of frames relative to adjacent frames.

According to another aspect of the present disclosure, the prediction model may be trained to classify two classes of motion vector fields corresponding to each of an x-axis and y-axis in a forward direction of the image frame by inputting the 2D cardiac ultrasound image.

In this case, the motion vector fields corresponding to each of the x-axis and y-axis in the backward direction of the image frame may be obtained based on the output value in the forward motion.

For example, parameter sharing may be performed in the layer before the output layer of the prediction model, so that when a reverse sequence for the forward motion is applied, it is possible to obtain motion vector fields corresponding to each of the x-axis and y-axis for the backward motion.

That is, motion estimation for both directions of the frame can be realized through this.

However, it is not limited thereto, and the prediction model may be trained to classify four classes of motion vector fields corresponding to the x-axis and y-axis of the forward and backward directions of the frame, respectively.

According to another embodiment of the present disclosure, the prediction model may be a model trained to derive correlations of frames corresponding to the resolutions for each of a plurality of frames having a plurality of resolutions, integrate the correlations, and estimate motion using information on the resolution of the multi-scale.

That is, through this, information collected at various resolutions may be effectively integrated to enable more precise motion estimation.

According to another embodiment of the present disclosure, the prediction model may be a model trained to determine feature maps for a plurality of frames corresponding to a large resolution to obtain information on the context, and estimate motion together with the context information and the motion features of the multi-scale resolution described above.

Accordingly, reliable motion estimation can be achieved through a clear distinction between the target heart area and the background area (noise) through motion estimation that incorporates context features.

In various embodiments of the present disclosure, the motion estimation unit of the prediction model may be applied with unsupervised learning-based learning. Furthermore, each of the target heart area segmentation unit and the cross-sectional view classification unit may be applied with supervised learning-based learning in which a specific area and each of the cross-sectional views in a specific frame or image are labeled.

In this case, the prediction model may be a semi-supervised learning model that can be selectively unsupervised or supervised learning depending on the learning purpose, such as prediction of a motion vector field and/or segmentation of an area and/or cross-sectional view classification.

In another embodiment of the present disclosure, the prediction model may be a model that has trained geometric modeling to determine a spline curve using a spline mathematical technique to estimate motion for the target heart area.

As used herein, the term “spline curve” is a smooth curve made up of data points that can be used to visually represent the target heart area in the cardiac ultrasound image.

In various embodiments of the present disclosure, the spline curve may be at least one of a B-spline curve, a Catmull-Rom spline curve, a Bezier curve, a non-uniform rational B-splines (NURBS) curve, a hypotrochoid curve, a hermite spline, and a parametric curve.

Preferably, the spline curve may be a B-spline curve, but is not limited thereto.

In various embodiments of the present disclosure, a spline surface may be generated that is made up of layers that include a time concept for the spline curve.

In more diverse embodiments of the present disclosure, a wall may be determined within the target heart area, an intermediate layer may be determined for the wall, a region of interest (ROI) may be determined by expanding the intermediate layer, and a spline curve may be generated for the determined ROI.

Here, when the endocardium and epicardium layers of the heart are determined based on the intermediate layer and the ROI is expanded, a highly accurate change analysis for the ROI is possible, and thus a highly reliable motion estimation result may be provided.

That is, since layered speckle tracking, which divides the cardiac muscle into multiple layers and individually analyzes the deformation of each layer, is possible, the function of the cardiac muscle may be evaluated in more detail.

In more diverse embodiments of the present disclosure, the generated spline curve may be post-processed based on curvature or weight.

In a specific example, in the case of a left ventricle (LV), since distortion of the spline curve may occur at the annulus point, which is the location of the annulus, which is a ring-shaped structure of the heart valve, a correction may be performed to perform curvature-based cutting that excludes data points whose curvature is equal to or more than a predetermined level.

In another specific example, in the LV, the apex, which is the lowest end portion of the heart, has a pointed shape, and weighted B-spline curve smoothing may be performed to reflect the structural characteristics of the apex well. More specifically, by reflecting a high weight on the data point and reducing the influence of the smooth penalty term, a smooth and detailed expression of the apex part with a high degree of curvature may be possible.

This may enable more precise contouring and motion estimation of target heart areas such as LV or LV wall.

According to one embodiment of the present disclosure, the prediction model may be a model trained to estimate motion based on 2D cardiac ultrasound images having sequences of 2 bits labeled for end systole (ES), end diastole (ED), mid (ES-ED) between end systole and end diastole, and mid (ED-ES) between end diastole and end systole.

In various embodiments, the prediction model may be based on at least one algorithm selected from among fully convolutional network (FCN) having DenseNet-121, U-net, VGG net, DenseNet, and encoder-decoder structure, deep neural network (DNN) such as SegNet, DeconvNet, and DeepLAB V3+, transformer such as Lawin+, SegFormer, and Swin, SqueezeNet, Alexnet, ResNet18, MobileNet-v2, GoogLeNet, Resnet-v2, Resnet50, RetinaNet, Resnet101, Inception-v3, HRNet, ResNeXt, and EfficientNet.

Furthermore, the prediction model may be an ensemble model based on at least two algorithm models among the aforementioned algorithms. However, it is not limited thereto.

As used in the present specification, the term “strain quantification parameter” refers to a strain parameter used to evaluate the cardiac function, which may include information on longitudinal deformation of the cardiac muscle.

In this case, the strain quantification parameter may be at least one of longitudinal strain indicating the degree of deformation of cardiac muscle in the longitudinal direction, radial strain indicating the degree of deformation of cardiac muscle in the radial direction, and circumferential strain indicating the degree of deformation of cardiac muscle in the circumferential direction.

In various embodiments, the strain quantification parameter may be, but is not limited to, longitudinal strain.

For example, the strain quantification parameter may be global longitudinal strain including a local minimum between end-diastole (ED) and aortic valve closure (AVC) in the cardiac cycle, a global minimum between ED and AVC, and a minimum throughout the cardiac cycle.

According to another embodiment of the present disclosure, the strain curve may be used to determine the strain quantification parameter for the LA.

Here, the strain curve for LA may represent reservoir, conduit, and contraction.

These mechanisms may be determined by extracting pre-A time based on electrocardiogram (ECG) data, but the accuracy may be very low.

Accordingly, in various embodiments of the present disclosure, the strain quantification parameter based on the strain curve may be determined, so that more accurate quantitative information on LA strain may be provided.

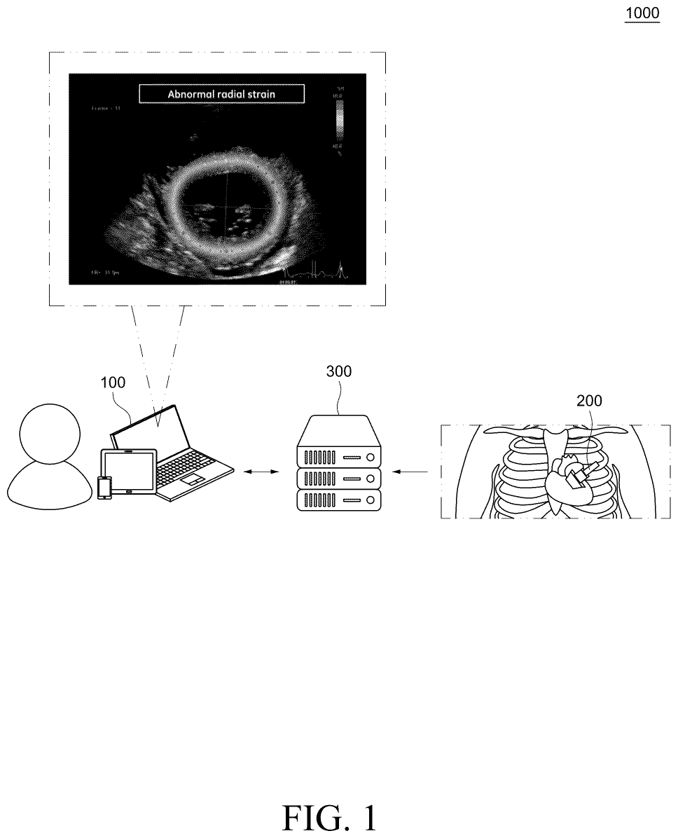

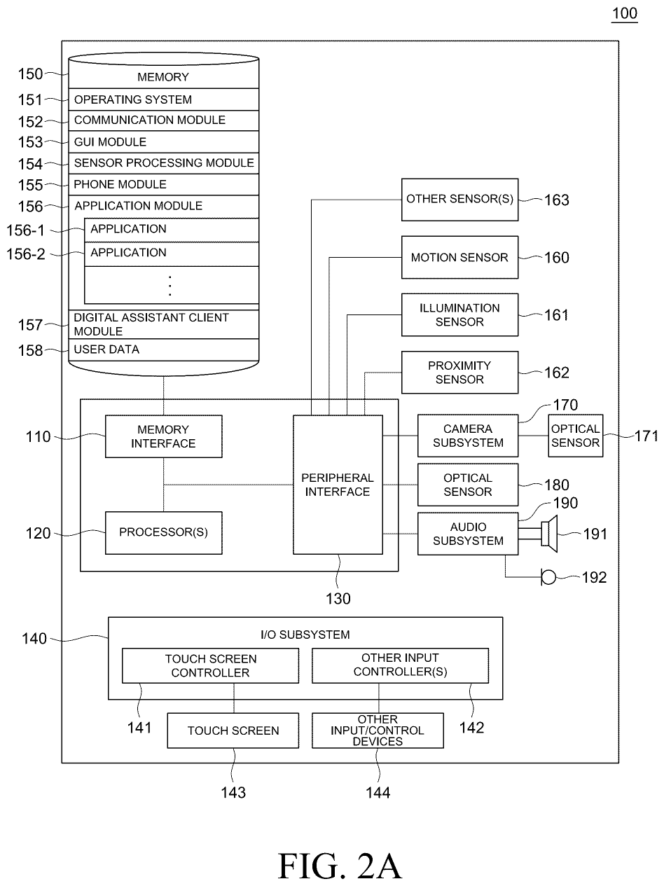

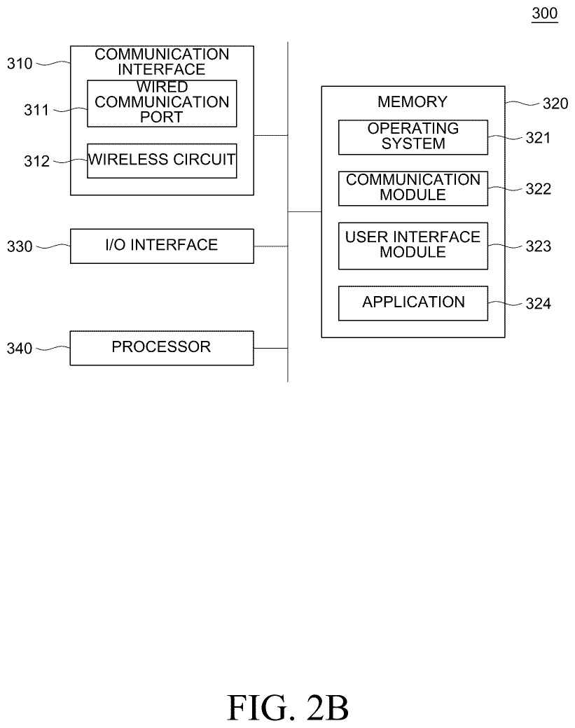

Hereinafter, a device for providing information on strain quantification according to various embodiments of the present disclosure will be described in detail with reference to FIGS. 1, 2A, and 2B.

FIG. 1 illustrates an information provision system for cardiac strain quantification using a device for providing information on cardiac strain quantification according to one embodiment of the present disclosure. FIG. 2A illustrates an exemplary configuration of a medical staff device that receives information on cardiac strain quantification according to one embodiment of the present disclosure. FIG. 2B illustrates an exemplary configuration of a server for providing information on cardiac strain quantification according to one embodiment of the present disclosure.

First, referring to FIG. 1, an information provision system 1000 may be a system configured to provide information related to cardiac strain quantification based on a cardiac ultrasound image of a subject. In this case, the information provision system 1000 may include a medical staff device 100 that receives information related to cardiac strain quantification, an ultrasound image diagnosis device 200 that provides a cardiac ultrasound image, and an information providing server 300 that generates information related to cardiac strain quantification based on the received cardiac ultrasound image.

In various embodiments of the present disclosure, information providing server 300 may be mounted on ultrasound image diagnosis device 200, and in this case, various information related to the measurement value may be displayed on the display unit (not illustrated) of ultrasound image diagnosis device 200.

That is, the user may use ultrasound image diagnosis device 200 to check information related to the cardiac strain quantification simultaneously with the diagnosis.

In various embodiments, medical staff device 100 is an electronic device that provides a user interface for displaying information related to cardiac strain quantification, and may include at least one of a smartphone, a tablet personal computer (PC), a laptop, and/or a PC.

Medical staff device 100 may receive the results of quantitative analysis of cardiac strain for the subject from information providing server 300 and may display the results received through the display unit (not illustrated).

Information providing server 300 may include a general-purpose computer, laptop, and/or data server, or the like, which perform various operations to determine information related to the cardiac strain quantification based on the cardiac ultrasound image provided from ultrasound image diagnosis device 200, such as an ultrasound diagnostic device. In this case, information providing server 300 may be a device for accessing a web server providing a web page or a mobile web server providing a mobile website, but is not limited thereto.

More specifically, information providing server 300 receives the cardiac ultrasound image from ultrasound image diagnosis device 200, performs segmentation and motion estimation of the target heart area within the received cardiac ultrasound image, and performs an operation to determine the strain quantification parameter based on the segmentation and the motion estimation. In this case, information providing server 300 may perform various functions for quantitative analysis of the cardiac strain from the cardiac ultrasound image using the prediction model.

Information providing server 300 may provide cardiac strain quantification parameters, masks for segmented target heart areas, classified cross-sectional views, or the like to medical staff device 100.

In this way, the information provided from information providing server 300 may be provided as a web page through a web browser installed on medical staff device 100, or may be provided in the form of an application or program. In various embodiments, such data may be provided in a form included in a platform in a client-server environment.

Next, with reference to FIGS. 2A and 2B, the components of information providing server 300 of the present disclosure will be specifically described.

First, with reference to FIG. 2A, medical staff device 100 may include a memory interface 110, one or more processors 120, and a peripheral interface 130. Various components within medical staff device 100 may be connected by one or more communication buses or signal lines.

Memory interface 110 may be connected to a memory 150 to transmit various data to one or more processors 120. Here, memory 150 may include at least one type of storage medium among flash memory type, hard disk type, multimedia card micro type, card type memory (for example, SD or XD memory, or the like), RAM, SRAM, ROM, EEPROM, PROM, network storage, cloud, and blockchain data.

In various embodiments, memory 150 may store at least one of an operating system 151, a communication module 152, a graphical user interface module (GUI) 153, a sensor processing module 154, a phone module 155, and an application module 156. Specifically, operating system 151 may include instructions for processing basic system services and instructions for performing hardware operations. Communication module 152 may communicate with at least one of other devices, computers, and servers. Graphical user interface module (GUI) 153 may process a graphical user interface. Sensor processing module 154 may process sensor-related functions (for example, processing voice input received using one or more microphones 192). Phone module 155 may process phone-related functions. Application module 156 may perform various functions of a user application, such as electronic messaging, web browsing, media processing, navigation, imaging, and other processing functions. In addition, medical staff device 100 may store one or more software applications 156-1 and 156-2 (for example, an information providing application) associated with a type of service in memory 150.

In various embodiments, memory 150 may store a digital assistant client module 157 (hereinafter, DA client module), and accordingly, may store instructions for performing functions of the client side of the digital assistant and various user data 158.

Meanwhile, DA client module 157 may obtain user's voice input, text input, touch input, and/or gesture input through various user interfaces (for example, I/O subsystem 140) provided in medical staff device 100.

In addition, DA client module 157 may output data in an audiovisual or tactile form. For example, DA client module 157 may output data composed of a combination of at least two or more of: voice, sound, notification, text message, menu, graphic, video, animation, and/or vibration. In addition, DA client module 157 may communicate with a digital assistant server (not illustrated) using a communication subsystem 180.

In various embodiments, DA client module 157 may collect additional information about the surroundings of medical staff device 100 from various sensors, subsystems, and peripheral devices to construct a context associated with the user input. For example, DA client module 157 may provide context information along with the user input to the digital assistant server to infer the user's intent. Here, the context information that may accompany the user input may include sensor information, such as lighting, ambient noise, ambient temperature, images of the surrounding environment, video, or the like. In another example, the context information may include the physical state of medical staff device 100 (for example, device orientation, device location, device temperature, power level, speed, acceleration, motion pattern, cellular signal strength, or the like). For another example, the context information may include information related to the software status of medical staff device 100 (for example, processes running on medical staff device 100, installed programs, past and present network activity, background services, error logs, resource usage, and the like).

In various embodiments, memory 150 may include additional or deleted instructions, and may further include additional configurations or exclude some configurations in addition to the configuration illustrated in FIG. 2A of medical staff device 100.

One or more processors 120 may control the overall operation of medical staff device 100 and may execute various commands to implement an interface that provides information related to the cardiac strain quantification by running applications or programs stored in memory 150.

One or more processors 120 may correspond to a computing device such as a central processing unit (CPU) or an application processor (AP). Additionally, one or more processors 120 may be implemented in the form of an integrated chip (IC) such as a system on chip (SoC) in which various computing devices such as a neural processing unit (NPU) are integrated.

Peripheral interface 130 may be connected to various sensors, subsystems, and peripheral devices, and may provide data so that medical staff device 100 can perform various functions. Here, it can be understood that the function performed by medical staff device 100 is performed by one or more processors 120.

Peripheral interface 130 may receive data from a motion sensor 160, a light sensor (illumination sensor) 161, and a proximity sensor 162, through which medical staff device 100 may perform orientation, light, and proximity detection functions, or the like. For another example, peripheral interface 130 may receive data from other sensors 163 (positioning system-GPS receiver, temperature sensor, biometric sensor), through which medical staff device 100 may perform functions related to other sensors 163.

In various embodiments, medical staff device 100 may include a camera subsystem 170 connected to peripheral interface 130 and an optical sensor 171 connected thereto, which enables medical staff device 100 to perform various photographing functions, such as taking pictures and recording video clips.

In various embodiments, medical staff device 100 may include communication subsystem 180 connected to peripheral interface 130. Communication subsystem 180 may be comprised of one or more wired/wireless networks, and may include various communication ports, radio frequency transceivers, and optical transceivers.

In various embodiments, medical staff device 100 includes an audio subsystem 190 coupled to a peripheral interface 130, audio subsystem 190 includes one or more speakers 191 and one or more microphones 192, such that medical staff device 100 can perform voice-activated functions, such as voice recognition, voice duplication, digital recording, and telephony.

In various embodiments, medical staff device 100 may include an I/O subsystem 140 coupled to peripheral interface 130. For example, I/O subsystem 140 may control a touch screen 143 included in medical staff device 100 via a touch screen controller 141. As an example, touch screen controller 141 may detect a user's contact and movement or cessation of contact and movement using any one of a plurality of touch sensing technologies, such as capacitive, resistive, infrared, surface acoustic wave technology, proximity sensor array, or the like. As another example, I/O subsystem 140 may control other input/control devices 144 included in medical staff device 100 via other input controller(s) 142. As an example, other input controller(s) 142 may control one or more buttons, rocker switches, thumb-wheels, infrared ports, USB ports, and pointer devices such as a stylus.

Next, referring to FIG. 2B, information providing server 300 may include a communication interface 310, a memory 320, an I/O interface 330, and a processor 340, and each component may communicate with each other through one or more communication buses or signal lines.

Communication interface 310 may be connected to medical staff device 100 and ultrasound image diagnosis device 200 through a wired/wireless communication network to exchange data. For example, communication interface 310 may receive the cardiac ultrasound image from ultrasound image diagnosis device 200, and transmit information on the cardiac strain quantification, that is, the strain quantification parameters, or data (for example, segmented mask) acquired during the process of determining the same to medical staff device 100.

Meanwhile, communication interface 310 that enables transmission and reception of such data includes a wired communication port 311 and a wireless circuit 312, in which wired communication port 311 may include one or more wired interfaces, such as Ethernet, universal serial bus (USB), FireWire, or the like. In addition, wireless circuit 312 may transmit and receive data with an external device via an RF signal or an optical signal. In addition, the wireless communication may use at least one of a plurality of communication standards, protocols, and technologies, such as GSM, EDGE, CDMA, TDMA, Bluetooth, Wi-Fi, VoIP, Wi-MAX, or any other suitable communication protocol.

Memory 320 may store various data used in information providing server 300. For example, memory 320 may store the cardiac ultrasound image, the prediction model modeled for quantitative analysis of cardiac strain from the cardiac ultrasound image, or various modules forming the prediction model, and further parameters for training these prediction models.

In various embodiments, memory 320 may include a volatile or nonvolatile storage medium capable of storing various data, commands, and information. For example, memory 320 may include at least one type of storage medium among flash memory type, hard disk type, multimedia card micro type, card type memory (such as SD or XD memory), RAM, SRAM, ROM, EEPROM, PROM, network storage, cloud, and blockchain data.

In various embodiments, memory 320 may store at least one configuration among an operating system 321, a communication module 322, a user interface module 323, and one or more applications 324.

Operating system 321 (for example, embedded operating systems such as LINUX, UNIX, MAC OS, WINDOWS, VxWorks, or the like) may include various software components and drivers for controlling and managing general system operations (for example, memory management, storage device control, power management, or the like) and may support communication between various hardware, firmware, and software components.

Communication module 322 may support communication with other devices through communication interface 310. Communication module 322 may include various software components for processing data received by wired communication port 311 or wireless circuit 312 of communication interface 310.

User interface module 323 may receive user requests or inputs from a keyboard, touch screen, microphone, or the like through I/O interface 330, and provide a user interface on the display.

Application 324 may include a program or module configured to be executed by one or more processors 340. Here, the application for providing information related to the cardiac strain quantification may be implemented on a server farm.

I/O interface 330 may connect an input/output device (not illustrated) of information providing server 300, for example, at least one of a display, a keyboard, a touch screen, and a microphone, to user interface module 323. I/O interface 330 may receive user input (for example, voice input, keyboard input, touch input, or the like) together with user interface module 323 and process a command according to the received input.

Processor 340 may be connected to communication interface 310, memory 320, and I/O interface 330 to control the overall operation of information providing server 300, and may perform various commands for providing information through an application or program stored in memory 320.

Processor 340 may correspond to a computing device such as a central processing unit (CPU) or an application processor (AP). In addition, processor 340 may be implemented in the form of an integrated chip (IC) such as a system on chip (SoC) in which various computing devices are integrated. Alternatively, processor 340 may include a module for calculating an artificial neural network model such as a neural processing unit (NPU).

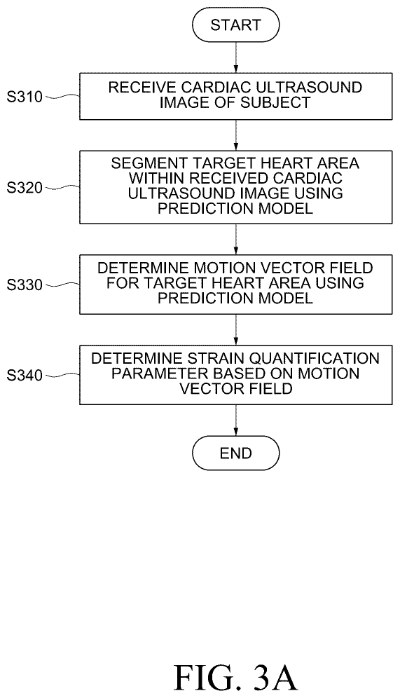

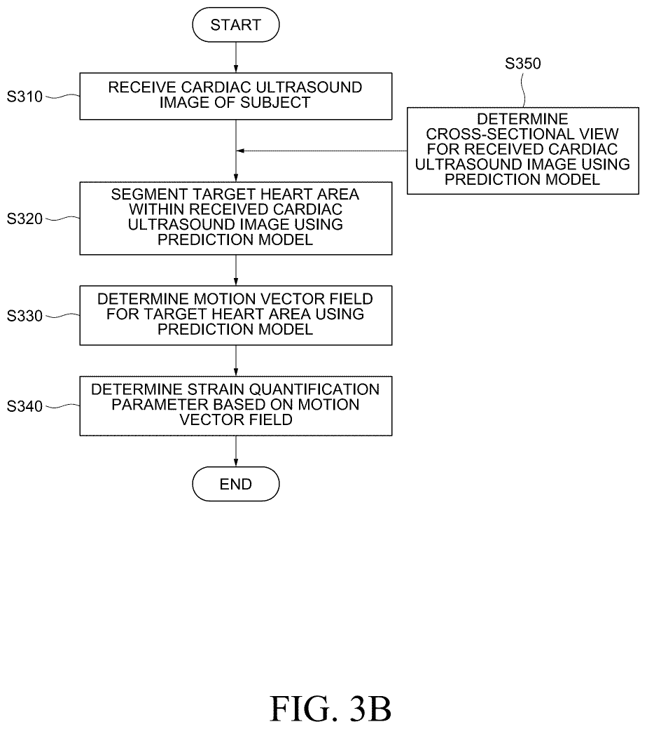

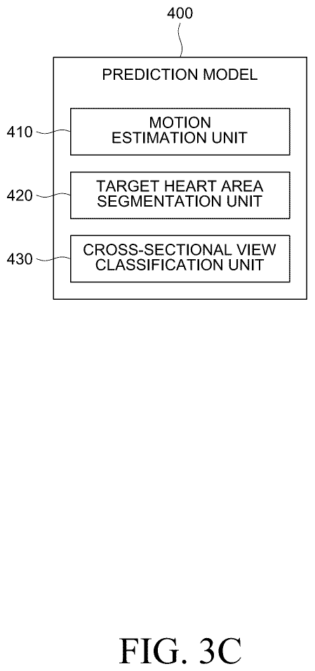

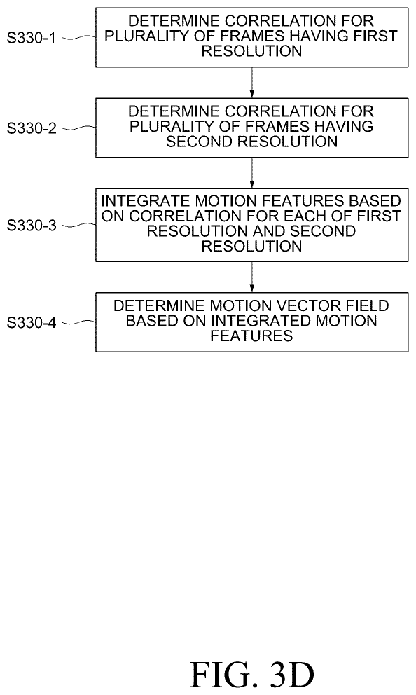

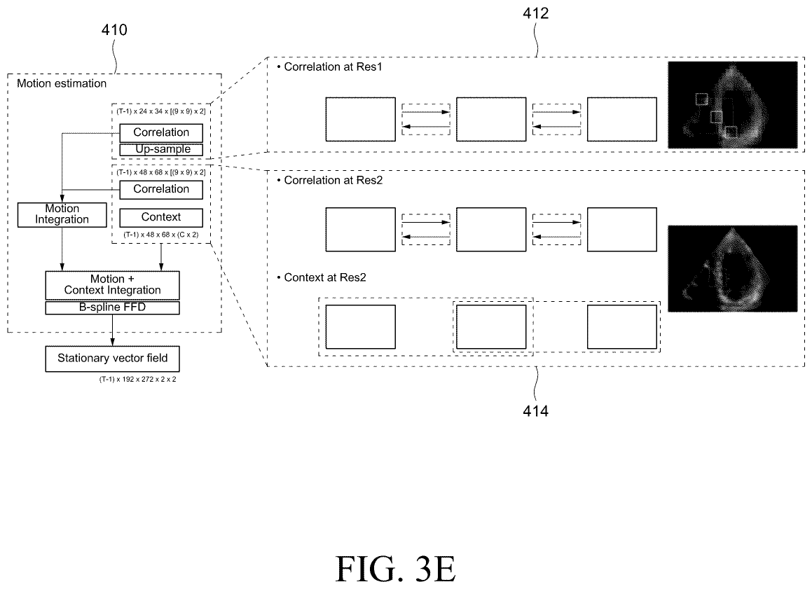

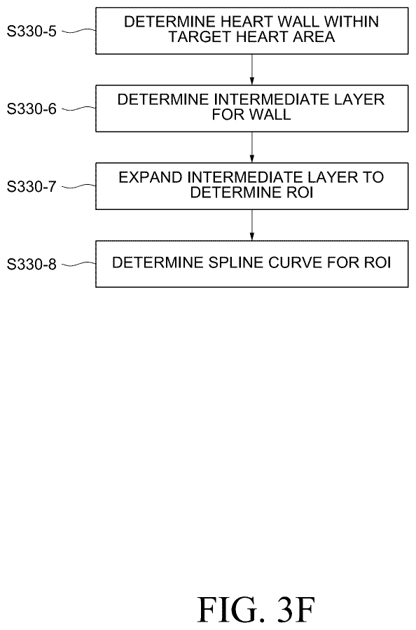

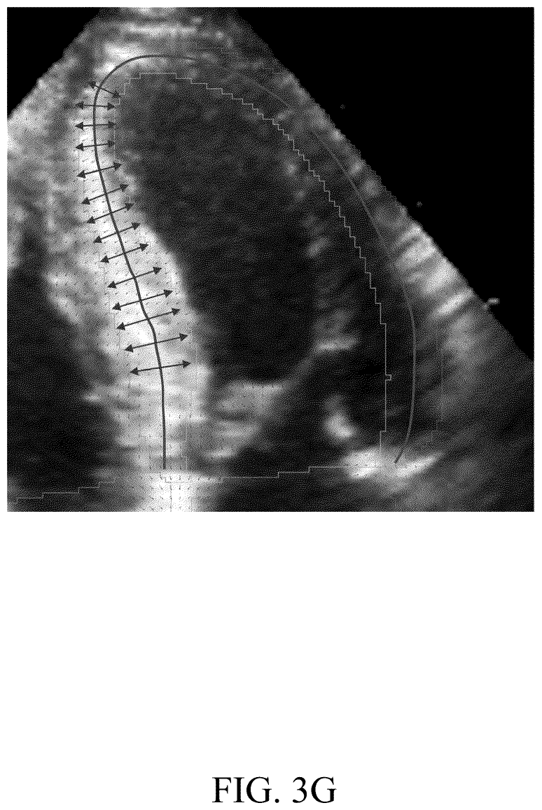

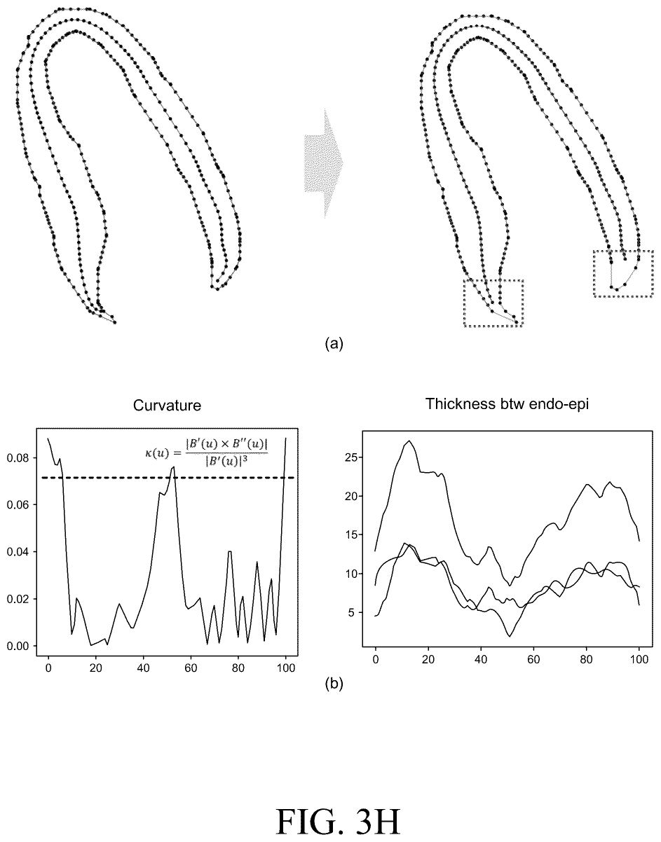

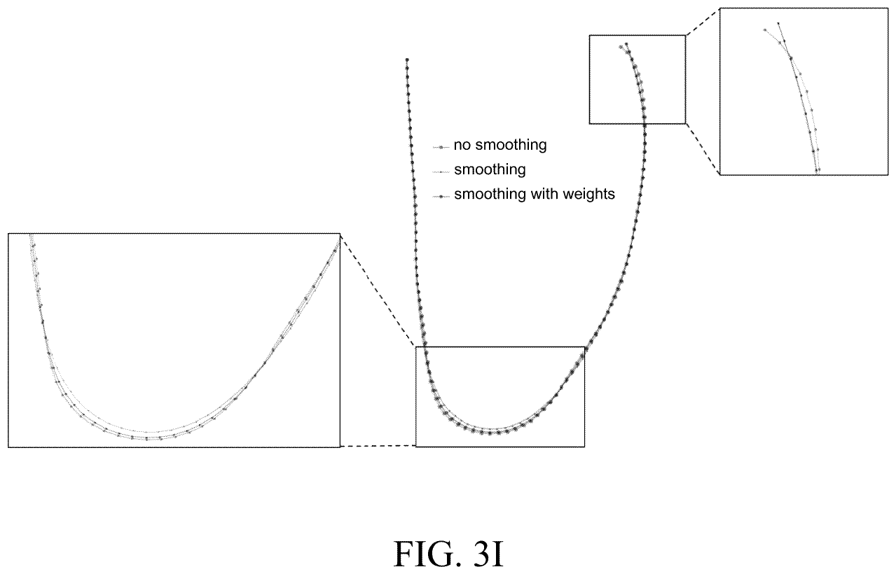

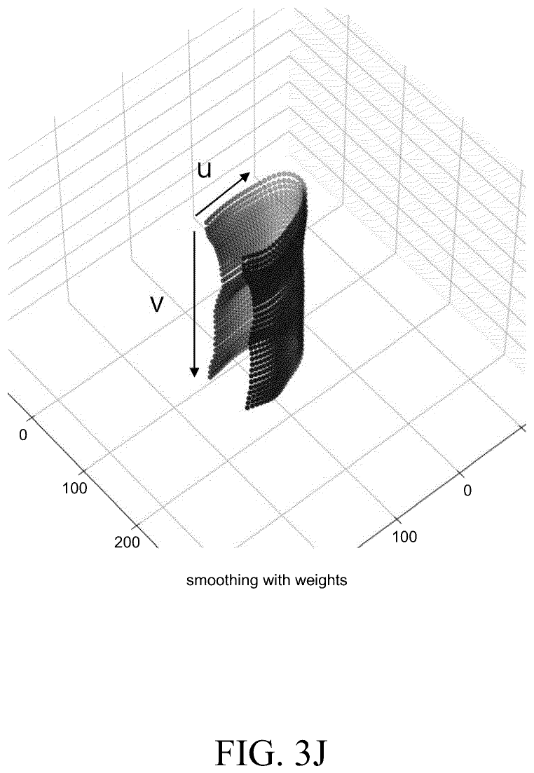

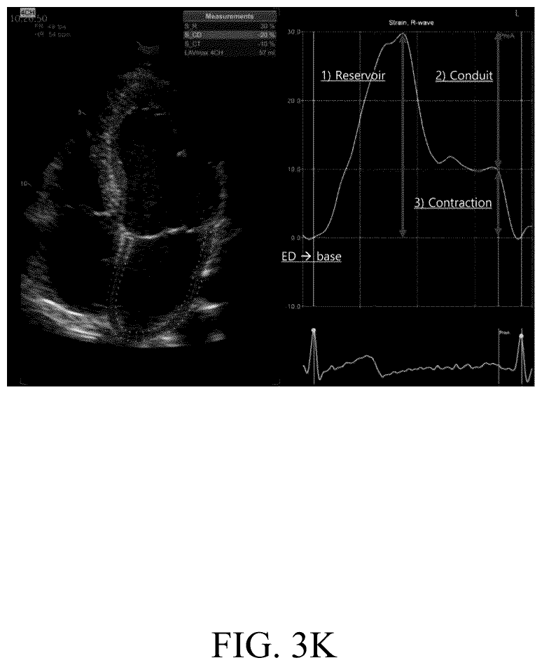

Hereinafter, a method for providing information on strain quantification according to one embodiment of the present disclosure will be described with reference to FIGS. 3A to 3K.

FIGS. 3A to 3K are schematic flowcharts for explaining the method for providing information on strain quantification based on a motion vector field in the device for providing information on strain quantification according to one embodiment of the present disclosure, and exemplarily illustrate a procedure for determining the motion vector field.

First, referring to FIG. 3A, the cardiac ultrasound image of the subject is received according to the method for providing information on strain quantification according to one embodiment of present disclosure (S310). Then, the target heart area is segmented using prediction model (S320), the motion vector field is determined (S330), and finally, the strain quantification parameter is determined based on motion vector field (S340).

More specifically, in step (S310) of receiving the cardiac ultrasound image, a 2D cardiac ultrasound image may be received.

For example, in step (S310) of receiving the cardiac ultrasound image, a 2D ultrasound image having a sequence of 2 bits may be received.

According to another aspect of the present disclosure, in step (S310) of receiving the cardiac ultrasound image, the cardiac ultrasound video consisting of the plurality of frames may be received.

In this case, the received cardiac ultrasound image may have at least one cross-sectional view among a B-mode parasternal long-axis (PLAX) cross-sectional view, a B-mode parasternal short-axis (PSAX) aortic valve (AV) level cross-sectional view, a B-mode PSAX mitral valve (MV) level cross-sectional view, a B-mode apical 4-chamber (A4C) cross-sectional view, a B-mode apical 2-chamber (A2C) cross-sectional view, a B-mode apical 3-chamber (A3C) cross-sectional view, a B-mode subcostal 4-chamber cross-sectional view, a B-mode suprasternal cross-sectional view, a B-mode left ventricular outflow tract (LVOT) cross-sectional view, and a B-mode right ventricular (RV) cross-sectional view for the cardiac ultrasound image. However, the present disclosure is not limited thereto, and the cardiac ultrasound image may also have cross-sectional views corresponding to the M-mode and Doppler mode.

Meanwhile, referring to FIG. 3B together, according to various embodiments of the present disclosure, a step (S350) of classifying a cross-sectional view of the received cardiac ultrasound image using the prediction model may be optionally performed after step (S310) of receiving the ultrasound image.

Then, the target heart area is segmented for the image whose cross-sectional view is classified using prediction model (S320).

According to an aspect of the present disclosure, the target heart area may be an area selected from a right ventricle anterior wall, a right ventricle (RV), an anterior wall of the aorta, an aorta, a posterior wall of an aorta, a left atrium (LA), and a posterior wall of a left atrium (LA).

According to another aspect of the present disclosure, a mask for the target heart area segmented by the prediction model may be output and provided to medical staff device 100.

Next, in step (S330) where the motion vector field is determined, the motion vector field for the target heart area of the cardiac ultrasound image is determined by the prediction model.

That is, the prediction model applied to various embodiments of the present disclosure may be a hybrid model in which various modules supporting the automation of strain quantification and image processing process are combined.

Referring to FIG. 3C, in one embodiment of the present disclosure, the prediction model may be composed of modules that perform multiple functions, such as a motion estimation unit 410 that estimates the motion vector field based on the input cardiac ultrasound image, a target heart area segmentation unit 420 that segments the target heart area, and a cross-sectional view classification unit 430 that classifies the cross-sectional view.

However, the present disclosure is not limited thereto, and the prediction model may be composed of modules with more diverse combinations depending on the purpose thereof.

For example, the prediction model may be composed of motion estimation unit 410 and target heart area segmentation unit 420, and the cardiac ultrasound image may be received by a cross-sectional view classification model that is independently separated from the prediction model, the cross-sectional view may be classified, and the result thereof may be transmitted to the prediction model.

The structural features of the prediction model used in various embodiments of the present disclosure will be specifically described later.

Referring again to FIG. 3A, in one embodiment of the present disclosure, in step (S330) where the motion vector field is determined, for at least one frame selected from among the plurality of frames, the motion vector field may be estimated based on an adjacent frame.

For example, in step (S330) where the motion vector field is determined, when the motion field is estimated in a forward direction, the motion vector field of a second frame I₂ following a first frame may be determined based on the motion field of the first frame I₁.

Furthermore, when the motion field is estimated in the backward direction, the motion vector field of a third frame I₀ preceding the first frame may be determined based on the motion field of the first frame I₁.

In this case, optionally, when a reverse sequence for the forward motion is applied, it may be possible to obtain a motion vector field corresponding to each of the x-axis and y-axis for the backward motion. That is, through this, motion estimation for both directions of the frame may be possible.

In another embodiment of the present disclosure, step (S330) in which a motion vector field is determined may be performed based on a multi-scale resolution.

More specifically, referring to FIG. 3D, a correlation for a plurality of frames having a first resolution is determined using a prediction model (S330-1), and a correlation for a plurality of frames having a second resolution higher than the first resolution is determined (S330-2). Then, based on the correlation for each of the first resolution and the second resolution, motion features are integrated (S330-3), and the motion vector field is determined based on the integrated motion features (S330-4).

Referring to FIG. 3E together, in step (S330-1) of determining correlations for the plurality of frames having the first resolution, features for the plurality of frames having the first resolution are determined (412) in motion estimation unit 410 of the prediction model, and in the step (S330-2) of determining correlations for the plurality of frames having the second resolution, the features for the plurality of frames having the second resolution are determined (414).

In this case, bidirectional feature extraction between pixels for adjacent frames in the plurality of frames having the first resolution is performed, and the correlations are determined (412), and bidirectional feature extraction between pixels for adjacent frames in the plurality of frames having the second resolution which is the higher resolution is performed and the correlations are determined (414).

Next, in step (S330-3) of integrating the motion features, the motion features for two resolutions are integrated (motion integration).

Then, in step (S330-4) where the motion vector field is determined, the context data of the feature map is extracted at the second resolution, which is the high resolution, and then the context data is used to estimate the motion together with the motion features of the integrated multi-scale resolution.

In this case, reliable and precise motion estimation can be made possible through a clear distinction between the target heart area and the background area (noise) through the motion estimation where the context features are fused.

Referring again to FIG. 3A, in step (S330) where the motion vector field is determined in one embodiment of the present disclosure, the contouring and spline curve of the target heart area based on the ROI are determined using the prediction model on which geometric modeling is performed, so that motion estimation can be made possible.

More specifically, referring to FIG. 3F, the heart wall is determined within segmented target heart area (S330-5), the intermediate layer is determined for heart wall (S330-6), the intermediate layer is expanded to determine the ROI for motion estimation (S330-7), and a spline curve for the ROI is determined (S330-8).

Referring to FIG. 3G, in step of determining ROI (S330-7), the intermediate layer is expanded to determine the ROI including the endocardium and epicardium layers of the heart. Accordingly, since high-precision analysis is possible in the motion analysis of the ROI of the target heart area, a highly reliable motion estimation result may be provided.

Returning to FIG. 3F, in step (S330-8) of determining the spline curve, the spline curve, which is a curve composed of data points, is generated.

That is, since layered speckle tracking is possible by dividing the cardiac muscles into multiple layers and individually analyzing the deformation of each layer, the function of the cardiac muscle may be evaluated in more detail.

Meanwhile, in step (S330-8) where the spline curve is determined, at least one spline curve among a B-spline curve, a Catmull-Rom spline curve, a Bezier curve, a non-uniform rational B-splines (NURBS) curve, a hypotrochoid curve, and a parametric curve may be generated.

Preferably, the spline curve may be a B-spline curve, but is not limited thereto.

Referring to FIGS. 3F and 3H together, according to various embodiments of the present disclosure, the spline surface composed of the plurality of layers including a time concept for the spline curve may be generated in step (S330-8) in which the spline curve is determined. That is, the spline surface may be generated by generating the spline curve multiple times.

In this case, the generated spline surface may be post-processed by going through smoothing processing.

According to another aspect of the present disclosure, after step (S330-8) in which the spline curve is determined, the generated spline curve may be post-processed based on the curvature or weight.

More specifically, referring to (a) and (b) of FIG. 3H, in the case of the LV, since distortion of the spline curve may occur at the annulus point, which is the location of the annulus, which is a ring-shaped structure of the heart valve, a correction may be performed to perform curvature-based cutting that excludes data points whose curvature is equal to or more than a predetermined level.

Referring to FIG. 3J, in the LV, the apex, which is the lowest end portion of the heart, has a pointed shape, and weighted B-spline curve smoothing may be performed to reflect the structural characteristics of the apex well. More specifically, different weights may be given to each data point, and in particular, lower weights may be applied to the apex so that the data points have a lower influence than other areas during the smoothing process. Accordingly, the curvature of the area at the annulus point may be straightened while the curvature of the apex may be maintained (see smoothing with weights).

Therefore, clearer contouring and motion estimation of target heart areas such as the LV or LV wall may be possible.

Referring back to FIG. 3A, in step (S340) where the strain quantification parameter is determined, at least one parameter among the longitudinal strain indicating the degree of deformation in the longitudinal direction of the cardiac muscle, the radial strain indicating the degree of deformation in the radial direction of the cardiac muscle, and the circumferential strain indicating the degree of deformation in the circumferential direction of the cardiac muscle may be determined.

According to various embodiments of the present disclosure, the strain curve may be used to determine the strain quantification parameter for the LA.

More specifically, referring together to FIG. 3K, the strain curve for the LA may represent reservoir, conduit, and contraction. These mechanisms may be determined by extracting pre-A time based on electrocardiogram (ECG) data, but the accuracy may be very low.

Accordingly, in various embodiments of the present disclosure, the strain quantification parameter based on the strain curve may be determined, so that more accurate quantitative information on LA strain may be provided.

According to the information provision method according to various embodiments of the present disclosure, various processes for strain analysis of not only LV but also LA, RV, and even RA may be performed using a hybrid prediction model based on geometric modeling.

That is, the present disclosure may provide the information provision system using an artificial neural network-based prediction model capable of analyzing various strain quantification parameters including not only the contractile function of the left ventricle but also the volume measurement of the left atrium and the diastolic function evaluation of the left ventricle.

Therefore, according to the present disclosure, it is possible to overcome the limitations of conventional strain quantitative analysis methods that are limited to measuring the size and volume of the left ventricle and calculating parameters such as LV ejection fraction for evaluating the contractile function of the left ventricle.

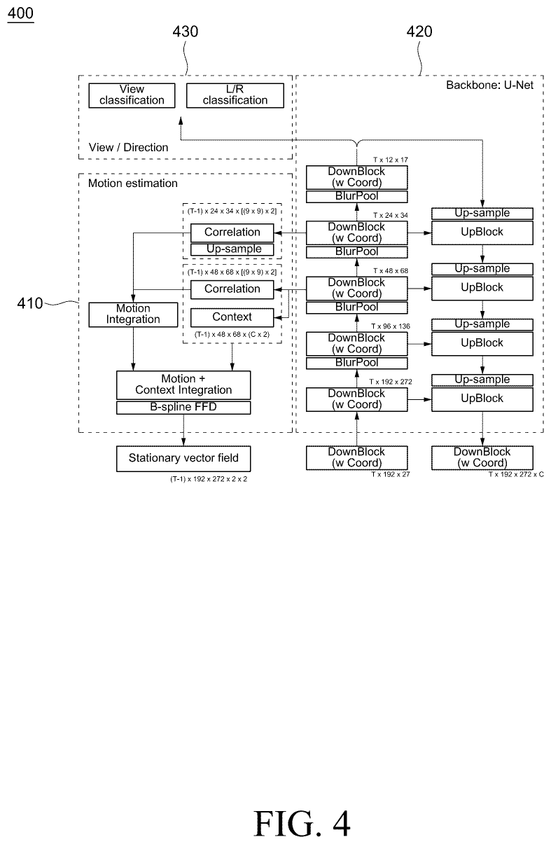

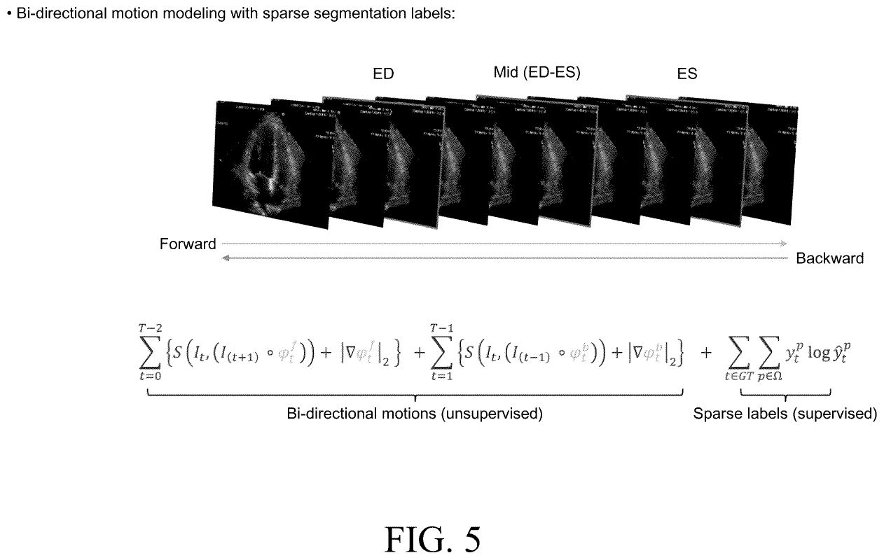

Hereinafter, the structure and learning method of the prediction model used in various embodiments of the present disclosure will be described with reference to FIGS. 4 and 5.

FIG. 4 illustrates an example of the structure of the prediction model applied to various embodiments of the present disclosure. FIG. 5 illustrates an example of the modeling procedure of the prediction model applied to various embodiments of the present disclosure.

First, referring to FIG. 4, a hybrid prediction model 400 may be composed of a plurality of modules, such as motion estimation unit 410 for estimating motion, target heart area segmentation unit 420 for segmenting the target heart area, and cross-sectional view classification unit 430.

More specifically, when the cardiac ultrasound video is input to target heart area segmentation unit 420 of a U-Net structure, features of the left area may be copied and pasted to the same level location of the right area through a line (skip connection) connected from the left block to the right block in the U-shaped segmentation unit. Through this, the features lost as input data decreases during prediction of the motion vector field may be corrected. Then, a finally segmented mask may be output through up-sampling.

That is, target heart area segmentation unit 420 of the structural features described above may be modeled to determine and efficiently learn the motion vector field between adjacent frames by considering the global structural features and regional feature similarity. Accordingly, it may provide highly reliable results for strain analysis that are robust to noise and regardless of image quality.

Next, through target heart area segmentation unit 420, some data during down-sampling processing for the input ultrasound image is transferred to motion estimation unit 410, and motion feature extraction for multi-scale resolution is performed therefrom. After that, the context data corresponding to the feature map of a large resolution and the integrated motion features for each resolution pass through the B-spline layer to determine the motion vector field for the target heart area.

In various embodiments of the present disclosure, the down-sampled data through target heart area segmentation unit 420 is transferred to cross-sectional view classification unit 430, and the cross-sectional view classification for the cardiac ultrasound image is classified based on this, and the direction of right or left is classified.

That is, using a single model of this hybrid structure, various processing for motion estimation may be performed on the cardiac ultrasound images.

Therefore, the present disclosure may overcome the problem of errors occurring due to multiple steps in strain manual measurement by providing information related to the strain quantification based on a single model.

Meanwhile, the structural characteristics of the prediction model are not limited to those described above and may have more diverse structures.

The prediction model used in various embodiments of the present disclosure may be trained to estimate the motion vector field through cyclic motion modeling.

More specifically, referring to FIG. 5, the prediction model may be used for training 2-bit units of cardiac ultrasound images labeled with the frames of the end systole (ES), the end diastole (ED), the mid (ES-ED) between the end systole and end diastole, and the mid (ED-ES) between the end diastole and end systole, respectively.

More specifically, when there are input data of a first set of 2-bit units and a second set different from the first set, in a case where it is desired to determine the motion vector field of the frame in the forward direction, the prediction model may be trained to determine the motion vector field of the first frame of the newly input second set based on the last frame of the first set. That is, the motion vector field of the first frame of the second set may be determined based on the motion field of the last frame of the first set that is not “0”.

Next, when it is desired to determine the motion vector field of the frame in the backward direction, the prediction model may be trained to determine the motion vector field of the last frame of the newly input second set based on the first frame of the first set.

In this way, the prediction model applied to various embodiments of the present disclosure may estimate the motion vector field by considering the spatiotemporal features of time-series cardiac ultrasound images through bi-directional cyclic motion modeling.

Meanwhile, the bi-directional cyclic motion modeling may be performed through Mathematical Expression 1 based on bidirectional circular motion, consistency constraints, and sparse labels for specific frames.

$$[ Mathematical Expression 1 ]$$

$${\underbrace{{\sum }_{t = 0}^{T − 2} \{ S ( {I}_{t} , ( {I}_{( t + 1 )} ∘ {φ}_{t}^{f} ) ) + {\vert ∇ {φ}_{t}^{f} \vert}_{2} \} + {\sum }_{t = 1}^{r − 1} \{ S ( {I}_{t} , ( {I}_{( t − 1 )} ∘ {φ}_{t}^{b} ) ) + {\vert ∇ {φ}_{t}^{b} \vert}_{2} \}}}_{Bi - directional motions ( unsupervised )} + {\underbrace{{\sum }_{t ∈ GT} {\sum }_{p ∈ Ω} {y}_{t}^{p} log {\hat{y}}_{t}^{p}}}_{Sparse labels ( supervised )}$$

Here,

$${φ}_{t}^{f}$$

is a motion vector estimated in the forward direction of a frame t, and

$${φ}_{t}^{b}$$

may mean a motion vector estimated in the backward direction of the frame t. Furthermore, y may mean a mask label, ŷ is a model prediction, and p may mean omega defined as an arbitrary point in the image space.

More specifically, the first equation (bi-directional cyclic motions) is a loss function that uses forward motion vectors and backward motion vectors to make (t+1)% T and (T−1)% T images match the present, and an equation that constrains the differential value of each motion vector to be close to 0, and the second equation (sparse labels) is an equation that applies a segmentation loss to the frame “t” that has the ground truth (GT) sparsely.

Through this modeling, the prediction model may be capable of bidirectional motion estimation based on the adjacent frame.

Meanwhile, in another embodiment of the present disclosure, the prediction model may be modeled by the following Mathematical Equation 2 for the diffeomorphic motion.

$$[ Mathematical Expression 2 ]$$

$$‖ x - ( {ϕ}^{- 1} ∘ ϕ ) ( x ) ‖ \approx 0$$

$$\frac{d {ϕ}^{( t )}}{dt} = v ( {ϕ}^{( t )} ) = v ∘ {ϕ}^{( t )}$$

In this case, (t) is parameterized in the [0,1] interval, and the stationary vector field (SVF) may be generated as a path of the diffeomorphic deformation field. Furthermore, in the initial state, it has an identity transformation ϕ⁽⁰⁾=ld, and SVF(v) may be integrated in the interval [0,1] (t).

Through this, it is possible to obtain a differentiable and inversely function-containing diffeomorphic deformation field in motion estimation, and it is possible to achieve a one-to-one correspondence and to preserve the spatial topology. Furthermore, the folding problem is minimized, and forward and backward simultaneous modeling is possible.

Meanwhile, in another embodiment of the present disclosure, the prediction model may be modeled for motion estimation based on a B-spline curve, and the B-spline curve may be defined by the following Mathematical Expression 3.

$$[ Mathematical Expression 3 ]$$

$$C = {\sum }_{i = 0}^{n} {N}_{i , d} ( u ) {P}_{i}$$

$$N \text{?} ( u ) = \{ \begin{matrix}1 & if u \text{?} \leq u < u \text{?} \\ 0 & otherwise\end{matrix}$$

$$N \text{?} ( u ) = \frac{u − \text{?}}{u \text{?} − u \text{?}} N \text{?} ( u ) + \frac{u \text{?} − u}{u \text{?} − u \text{?}} N \text{?} ( u )$$

$$\text{?} \text{indicates text missing or illegible when filed}$$

Here, N_{i,d}(u) is a basis function, and Pᵢ may be a data point which is a control point.

Meanwhile, in another embodiment of the present disclosure, the prediction model may be modeled to perform smoothing on a B-spline curve by the following Mathematical Expressions 4 and 5.

$$[ Mathematical Expression 4 ]$$

$$\begin{matrix}PSS = \sum \text{?} {( {y}_{d} - C ( u ) )}^{2} + λ \int {( C \text{?} ( u ) )}^{2} du \\ {= \sum \text{?} {y}_{d} - \sum \text{?} N \text{?} ( {u}_{d} ) P \text{?} )}^{2} + λ \int {( \sum \text{?} N \text{?} ( u ) P \text{?} )}^{2} du\end{matrix}$$

$$[ Mathematical Expression 5 ]$$

$$PSS = {( Y - NP )}^{T} ( Y - NP ) + λ {P}^{T} Ω P$$

$$∴ P = {( {N}^{T} N + λΩ )}^{- 1} {N}^{T} Y$$

$$N = ( \begin{matrix}{N}_{0 , p} ( {u}_{1} ) & ⋯ & {N}_{n , p} ( {u}_{1} ) \\ ⋮ & ⋱ & ⋮ \\ {N}_{0 , p} ( {u}_{D} ) & ⋯ & {N}_{n , p} ( {u}_{D} )\end{matrix} ) \text{}$$

$$\text{} P = ( \begin{matrix}{P}_{0} \\ ⋮ \\ {P}_{n}\end{matrix} )$$

$$\text{} {( {\sum }_{i = 0}^{n} {N}_{i , p}^{″} ( u ) {P}_{i} )}^{2} = {( {N}^{′′} P )}^{T} ( {N}^{′′} P ) = {P}^{T} Ω P$$

$$\text{?} \text{indicates text missing or illegible when filed}$$

Here, Mathematical Expression 4 is an equation that reflects a penalty term to minimize the curvature of a curve in a B-spline curve fitting process, and Mathematical Expression 5 may be an equation that expands Mathematical Expression 4 into a matrix form.

In particular, the prediction model may be modeled to perform weight-based smoothing on a B-spline curve by the following Mathematical Expression 6.

$$[ Mathematical Expression 6 ]$$

$$\begin{matrix}WPSS = {\sum }_{d = 1}^{D} {{w}_{d} ( {y}_{d} - C ( u ) )}^{2} + λ \int {( {C}^{″} ( u ) )}^{2} d u \\ = {\sum }_{d = 1}^{D} {{w}_{d} ( {y}_{d} - {\sum }_{i = 0}^{n} {N}_{i , p} ( {u}_{d} ) {P}_{i} )}^{2} + λ \int {( {\sum }_{i = 0}^{n} {N}_{i , p}^{″} ( u ) {P}_{i} )}^{2} du\end{matrix}$$

$$WPSS = {( Y - N P )}^{T} {W}^{T} W ( Y - NP ) + λ {P}^{T} Ω P$$

$$∴ P = {( {N}^{T} {W}^{T} WN + λΩ )}^{- 1} {N}^{T} {W}^{T} WY$$

$${W}^{T} W = ( \begin{matrix}{( {w}_{1} )}^{2} & ⋯ & 0 \\ ⋮ & ⋱ & ⋮ \\ 0 & ⋯ & {( {w}_{D} )}^{2}\end{matrix} )$$

Here, Mathematical Expression 6 is an equation that is set to alleviate distortion resulting from the calculation of Mathematical Expression 4 by increasing the weight of data points in cases where there is a place with a large curvature, such as the apex of the LV.

Through this mathematical modeling, the prediction model may accurately estimate the motion for the generated spline curve. Meanwhile, the modeling of the prediction model is not limited to the above-described method and may be performed through various methods.

### Evaluation: Diagnostic Performance Evaluation of Device for Providing Information on Strain Quantification

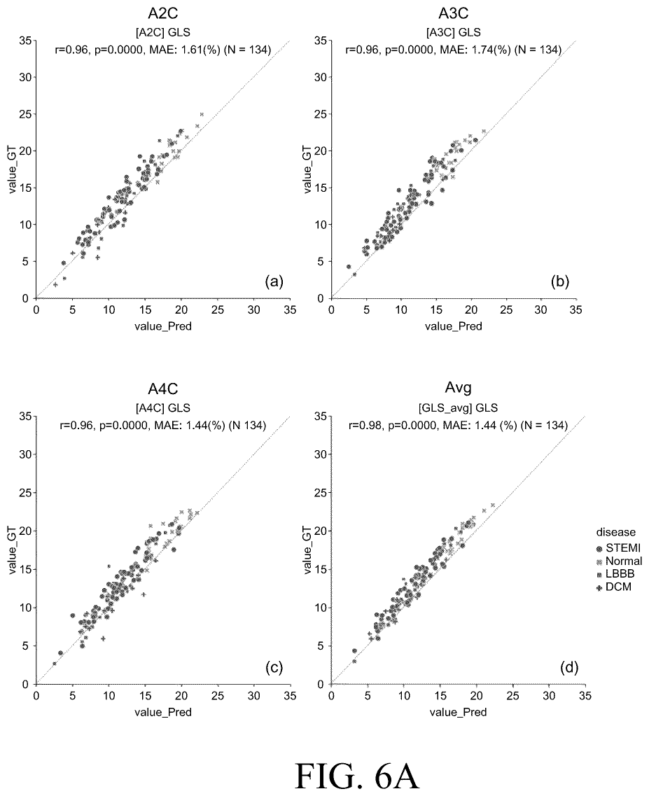

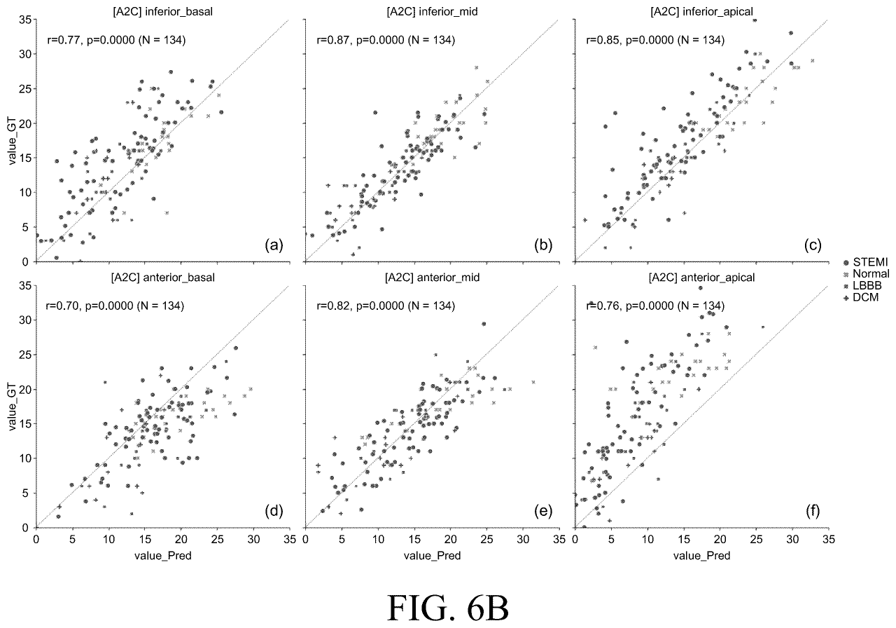

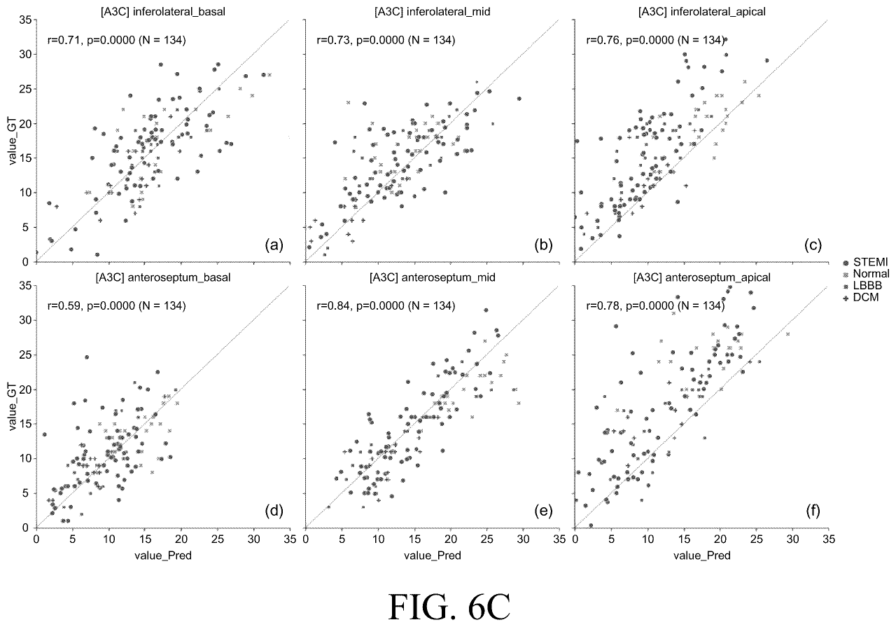

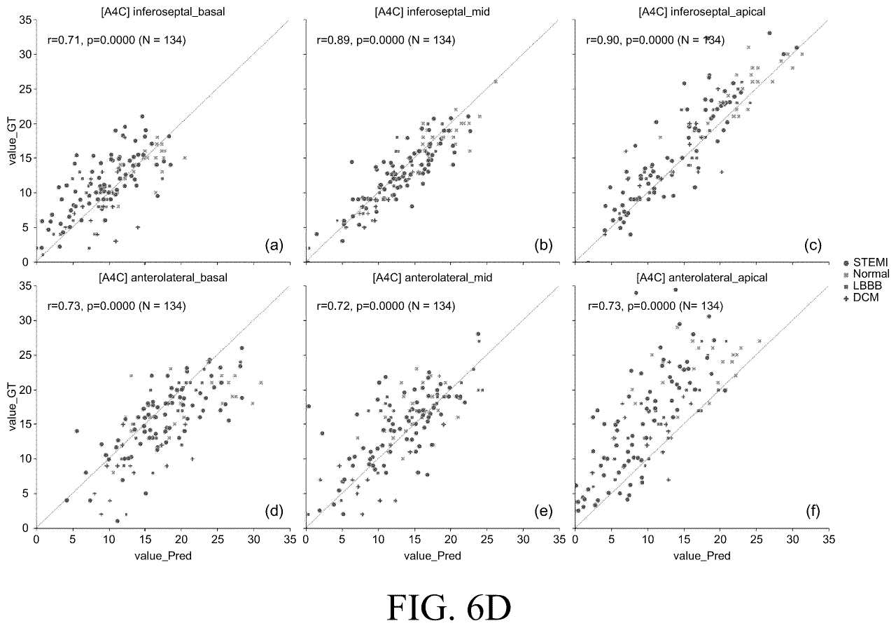

Hereinafter, with reference to FIGS. 6A to 6D, FIGS. 7 and 8, the evaluation results of the prediction model applied to the device for providing information on strain quantification according to various embodiments of the present disclosure are described.

More specifically, with reference to FIG. 6A, the results of deriving strain quantification parameters of GLS for the cardiac ultrasound images corresponding to A2C, A3C, and A4C cross-sectional views of the LV using the prediction model are illustrated.

Furthermore, with reference to FIGS. 6B to 6D, the evaluation results for motion-based strain quantification are illustrated.

In all results, the prediction model illustrates results with a slope close to 1 in both the heart disease onset subjects such as STEMI (ST-elevation myocardial infarction) onset subject, LBBB (left bundle branch block) subject, and DCM (dilated cardiomyopathy) subject, and the normal subjects, as the predicted values are very similar to the actual values under various conditions and seeds.

This suggests that the prediction performance of the strain quantification parameters including GLS is excellent and stable.

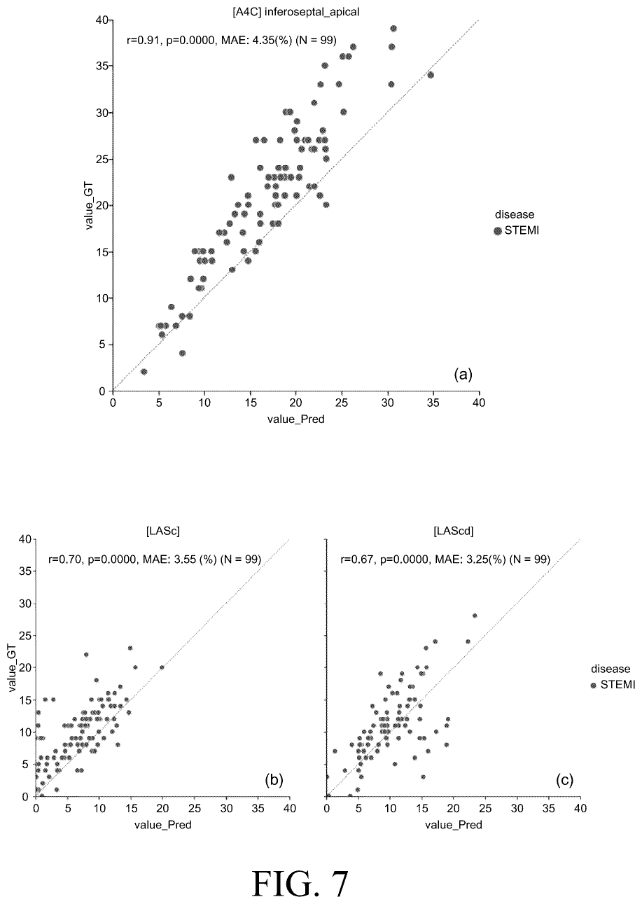

Next, referring to (a) of FIG. 7, the evaluation results for the LA strain quantification parameters of the STEMI subjects using the prediction model also illustrate excellent prediction performance.

In contrast, referring to (b) and (c) of FIG. 7, when EchoPAC which is the conventional cardiac ultrasound image analysis software is used, the diagnostic performance is somewhat reduced as the predicted values for the LA strain quantification parameters are different from the actual values.

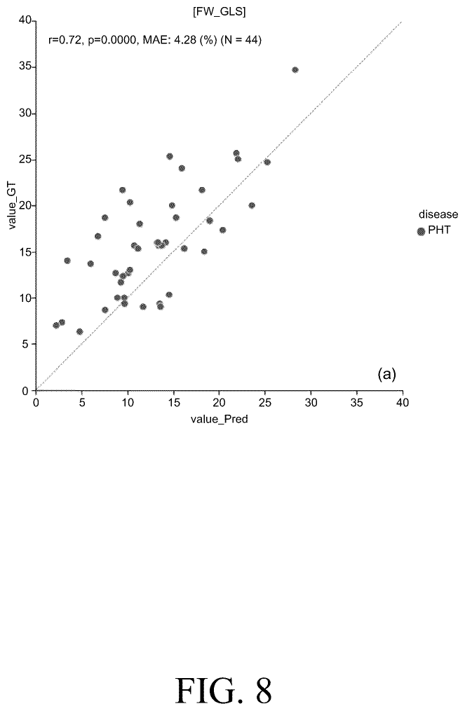

Next, referring to FIG. 8, the evaluation results for the RV strain quantification parameters of the PHT (pulmonary hypertension) object using the prediction model illustrate that the prediction performance has a Pearson correlation coefficient of r of 0.72.

According to the information provision method according to the various embodiments of the present disclosure above, various processes for strain analysis of not only the LV but also the LA, RV, and even the RA may be performed using the hybrid prediction model based on geometric modeling.

That is, the present disclosure may provide the information provision system using an artificial neural network-based prediction model capable of analyzing various strain quantification parameters including not only the contractile function of the left ventricle but also the volume measurement of the left atrium and the diastolic function evaluation of the left ventricle.

Therefore, according to the present disclosure, it is possible to overcome the limitations of conventional strain quantitative analysis methods that are limited to measuring the size and volume of the left ventricle and calculating parameters such as LV ejection fraction for evaluating the contractile function of the left ventricle.

That is, the present disclosure may contribute to early diagnosis of diseases and good treatment prognosis by providing information on strain quantification.

Although the embodiments of the present disclosure have been described in more detail with reference to the attached drawings, the present disclosure is not necessarily limited to these embodiments, and various modifications may be implemented within a scope that does not deviate from the technical idea of the present disclosure. Therefore, the embodiments disclosed in the present disclosure are not intended to limit the technical idea of the present disclosure but to explain it, and the scope of the technical idea of the present disclosure is not limited by these embodiments. Therefore, the embodiments described above should be understood as illustrative and not restrictive in all respects. The scope of protection of the present disclosure should be interpreted by the claims below, and all technical ideas within the equivalent scope should be interpreted as being included in the scope of rights of the present disclosure.

What is claimed is:

1. A method for providing information on strain quantification implemented by a processor, the method comprising:
receiving a cardiac ultrasound image including a target heart area of a subject;
determining a motion vector field for the target heart area in the received cardiac ultrasound image using a prediction model trained to segment the target heart area using the cardiac ultrasound image as an input and determine the motion vector field based on the segmented target heart area; and
determining a strain quantification parameter based on the motion vector field,
wherein the target heart area is at least one of a left ventricle (LV), a right ventricle (RV), a left atrium (LA), and a right atrium (RA).

2. The method according to claim 1, wherein the cardiac ultrasound image is a video including a plurality of frames, and
the prediction model is configured to determine the motion vector field for at least one frame selected from the plurality of frames based on a frame adjacent to the at least one frame.

3. The method according to claim 2, wherein the plurality of frames includes a plurality of frames having a first resolution and a plurality of frames having a second resolution for the target heart area, and
the determining of the motion vector field includes, by using the prediction model,
determining a correlation for the plurality of frames having the first resolution,
determining a correlation for the plurality of frames having the second resolution,
integrating a motion feature based on the correlation for each of the first resolution and the second resolution, and
determining the motion vector field based on the integrated motion feature.

4. The method according to claim 3, wherein the first resolution or the second resolution has a resolution greater than that of a remaining one, and
the determining of the motion vector field based on the integrated motion feature further includes
determining a feature map for a plurality of frames having the resolution greater than that of the remaining one, and
determining the motion vector field based on the feature map and the integrated motion feature.

5. The method according to claim 2, wherein the plurality of frames includes a first frame for the target heart area and a second frame that is a frame before or after the first frame, and
the determining of the motion vector field includes, by using the prediction model,
determining a first motion vector field for the first frame, and
estimating a second motion vector field for the second frame based on the first motion vector field.

6. The method according to claim 1, wherein the determining of the motion vector field includes, by using the prediction model, determining a spline curve using a spline mathematical technique to estimate motion for the target heart area.

7. The method according to claim 6, wherein the determining of the spline curve further includes
determining a heart wall within the target heart area,
determining an intermediate layer for the heart wall,
expanding the intermediate layer to determine a region of interest (ROI), and
obtaining the spline curve for the ROI.

8. The method according to claim 6, wherein the determining of the spline curve includes determining a plurality of spline curve layers to obtain a spline surface including the plurality of spline curve layers.

9. The method according to claim 6, further comprising correcting the determined spline curve.

10. The method according to claim 9, wherein the correcting of the spline curve includes
determining a curvature for the spline curve, and
correcting the spline curve by cutting the spline curve by excluding a data point, the curvature of which is equal to or greater than a predetermined level, among data points forming the spline curve.

11. The method according to claim 9, wherein the correcting of the spline curve further includes a smoothing by assigning weight to a data point corresponding to a specific area of the target heart area in a process of generating the spline curve.

12. The method according to claim 1, wherein the prediction model is a model further trained to classify a cross-sectional view of the ultrasound image using the cardiac ultrasound image as the input, and
the determining of the motion vector field further includes, by using the prediction model,
classifying a cross-sectional view of the received ultrasound image,
segmenting the target heart area for the ultrasound image corresponding to the classified cross-sectional view, and
determining the motion vector field for the target heart area, and
the determining of the strain quantification parameter further includes determining the strain quantification parameter corresponding to the classified view.

13. The method according to claim 1, further comprising outputting and providing a mask for the target heart area segmented by the prediction model.

14. The method according to claim 1, wherein the target heart area is LA, and
the determining of the strain quantification parameter includes
determining a strain curve for the LA based on the motion vector field, and
determining a quantification parameter for the LA based on the strain curve.

15. A device for providing information on strain quantification, the device comprising:
a communication unit configured to receive a cardiac ultrasound image including a target heart area of a subject; and
a processor functionally connected to the communication unit,
wherein the processor is configured to
determine a motion vector field for the target heart area in the received cardiac ultrasound image using a prediction model trained to segment the target heart area using the cardiac ultrasound image as an input and determine the motion vector field based on the segmented target heart area, and
determine a strain quantification parameter based on the motion vector field, and
the target heart area is at least one of a left ventricle (LV), a right ventricle (RV), a left atrium (LA), and a right atrium (RA).

16. The device according to claim 15, wherein the cardiac ultrasound image is a video including a plurality of frames, and
the prediction model is configured to determine the motion vector field for at least one frame selected from the plurality of frames based on a frame adjacent to the at least one frame.

17. The device according to claim 16, wherein the plurality of frames includes a plurality of frames having a first resolution and a plurality of frames having a second resolution for the target heart area, and
the processor is further configured to, by using the prediction model,
determine a correlation for the plurality of frames having the first resolution,
determine a correlation for the plurality of frames having the second resolution,
integrate a motion feature based on the correlation for each of the first resolution and the second resolution, and
determine the motion vector field based on the integrated motion feature.

18. The device according to claim 17, wherein the first resolution or the second resolution has a resolution greater than that of a remaining one, and
the processor is further configured to
determine a feature map for a plurality of frames having the resolution greater than that of the remaining one, and
determine the motion vector field based on the feature map and the integrated motion feature.

19. The device according to claim 16, wherein the plurality of frames includes a first frame for the target heart area and a second frame that is a frame before or after the first frame, and
the processor is further configured to, by using the prediction model,
determine a first motion vector field for the first frame, and
estimate a second motion vector field for the second frame based on the first motion vector field.

20. The device according to claim 15, wherein the processor is further configured to, by using the prediction model, determine a spline curve using a spline mathematical technique to estimate motion for the target heart area.
